(19) United States
(12) Patent Application Publication
(10) Pub. No.: US 2018/0153835 A1
(43) Pub. Date: Jun. 7, 2018

(54) COMPOSITIONS OF DICLOFENAC ACID

(71) Applicant: Lupin Limited, Mumbai (IN)

(72) Inventors: Sagar T. Shinde, Pune (IN); Dhanashree B. Mistry, Pune (IN); Subhash Gore, Pune (IN); Vijaya T. Kumar, Pune (IN)

(21) Appl. No.: 15/579,632

(22) PCT Filed: May 30, 2016
(86) PCT No.: PCT/IB2016/053176

§ 371 Date: Dec. 5, 2017

(30) Foreign Application Priority Data
Jun. 5, 2015 (IN) 2176/MUM/2015

(51) Int. Cl. A61K 31/196; A61K 9/00; A61K 9/14; A61K 9/16; A61K 9/51; A61K 47/22; A61K 47/26; A61P 29/00

(52) U.S. Cl. CPC A61K 31/196; A61K 9/0053; A61K 9/141; A61K 9/1605; A61P 29/00; A61K 9/5192; A61K 47/22; A61K 47/26; A61K 9/1682

(57) ABSTRACT

The present invention provides solid oral pharmaceutical compositions comprising wet milled Diclofenac acid and one or more pharmaceutically acceptable excipients and process for preparation thereof. The present invention particularly relates to solid oral pharmaceutical compositions comprising wet milled diclofenac acid with median particle size of less than 1000 nm. In addition the compositions of present invention have the comparable dissolution profiles with marketed composition of diclofenac acid. Maximum Plasma Concentration (Cₘₐₓ) and Area Under Curve (AUC) values of compositions of present invention are within the limit of 80% to 125% of Cₘₐₓ and AUC of the marketed composition of diclofenac acid capsules respectively.

### FIELD OF THE INVENTION

The present invention relates to solid oral pharmaceutical compositions comprising wet milled Diclofenac acid and one or more pharmaceutically acceptable excipients and process for preparation thereof.

### BACKGROUND OF INVENTION

Non-steroidal anti-inflammatory drugs (NSAIDs) are a backbone in the management of acute and chronic pain and inflammation treatments in both parenteral and oral dosage forms. Oral dosages range from 100-200 mg/day, while parenteral dosages range from 75-150 mg/day (1-2 mg/kg/day) by either infusion or intermittent (divided) doses.

Diclofenac or 2-[(2, 6-dichlorophenyl) amino] benzene acetic acid belongs to a class of NSAIDs and is useful for its anti-inflammatory, analgesic and antipyretic actions in treatment of acute and chronic pain and inflammation. It is marketed as injection, oral immediate release tablets, capsules, sustained release tablets and topical formulations.

Diclofenac acid is currently approved in the United States for the treatment of mild to moderate acute pain and Osteoarthritis pain in adults. It is commercially marketed by Iroko Pharms LLC in the form of capsules of 18 mg and 35 mg strengths under the trade name ZORVOLEX®.

U.S. Pat. No. 8,679,544 discloses methods for producing particles of diclofenac using dry milling processes as well as compositions comprising dry milled diclofenac. It is further disclosed that wet grinding is not suitable for particle size reductions as flocculation restricts the lower particle size limit to approximately 10 microns (10,000 nm). The wet milling process is also prone to contamination, thereby leading to a bias in the pharmaceutical art against wet milling. Also it discloses the problems associated with wet milling such as the mill blockage, low yield because of caking which makes wet milling techniques incompetent of being applied to poorly water-soluble biologically active materials like diclofenac.

However, the inventors of the present invention surprisingly found that a solid oral pharmaceutical composition comprising diclofenac acid with median particle size of less than 1000 nm can be prepared by wet milling process. Further, such compositions are found to be bioequivalent to the marketed composition of diclofenac acid capsules.

### SUMMARY OF THE INVENTION

The present invention provides solid oral pharmaceutical compositions comprising wet milled Diclofenac acid and one or more pharmaceutically acceptable excipients and process for preparation thereof. The present invention particularly relates to solid oral pharmaceutical compositions comprising wet milled diclofenac acid with median particle size of less than 1000 nm. In addition the compositions of present invention have the comparable dissolution profiles with marketed composition of diclofenac acid. Maximum Plasma Concentration (Cₘₐₓ) and Area Under Curve (AUC) values of compositions of present invention are within the limit of 80% to 125% of Cₘₐₓ and AUC of the marketed composition of diclofenac acid capsules respectively.

### OBJECTIVE OF THE INVENTION

The main object of the invention is to provide solid oral pharmaceutical compositions comprising wet milled diclofenac acid and one or more pharmaceutically acceptable excipients wherein the diclofenac acid having median particle size of less than 1000 nm.

Another object of the invention is a process of preparing a solid oral pharmaceutical composition comprising wet milled diclofenac acid and one or more pharmaceutically acceptable excipients wherein the diclofenac acid having median particle size of less than 1000 nm

Another object of the invention is to provide a solid oral pharmaceutical composition comprising wet milled diclofenac acid and one or more pharmaceutically acceptable excipients wherein the diclofenac acid having median particle size of less than 1000 nm, such that when the composition tested in-vitro by USP Apparatus I (Basket) method of U.S. Pharmacopoeia at 100 rpm, at 37° C. in 900 ml of 0.05% sodium lauryl sulfate in citric acid solution buffered to pH 5.5 exhibit diclofenac acid release rate of at least 80% by weight within 10 minutes.

Another object of the invention is to provide a solid oral pharmaceutical composition comprising wet milled diclofenac acid and one or more pharmaceutically acceptable excipients wherein the diclofenac acid having median particle size of less than 1000 nm, such that Maximum Plasma Concentration (Cₘₐₓ) and Area Under Curve (AUC) values of the compositions are within the limit of 80% to 125% of Cₘₐₓ and AUC of the marketed composition of diclofenac acid capsule respectively.

Another object of the invention is to provide a method of treating pain comprising administering the solid oral pharmaceutical composition comprising wet milled diclofenac acid and one or more pharmaceutically acceptable excipients wherein the diclofenac acid having median particle size of less than 1000 nm.

### DETAILED DESCRIPTION OF THE INVENTION

The main embodiment of invention is to provide solid oral pharmaceutical compositions comprising wet milled Diclofenac acid and one or more pharmaceutically acceptable excipients and a process for preparation thereof.

In another embodiment, a solid oral pharmaceutical composition comprising wet milled diclofenac acid and one or more pharmaceutically acceptable excipients.

In another embodiment, a solid oral pharmaceutical composition in the form of capsule comprising wet milled diclofenac acid and one or more pharmaceutically acceptable excipients.

In another embodiment, a solid oral pharmaceutical composition comprising wet milled diclofenac acid and one or more pharmaceutically acceptable excipients, wherein the diclofenac acid having median particle size of less than 1000 nm.

In another embodiment, a solid oral pharmaceutical composition comprising wet milled diclofenac acid and one or more pharmaceutically acceptable excipients, wherein the diclofenac acid having median particle size of less than 500 nm.

In another embodiment, a solid oral pharmaceutical composition in the form of capsule comprising wet milled diclofenac acid and one or more pharmaceutically acceptable excipients, wherein the diclofenac acid having median particle size of less than 1000 nm.

In another embodiment, a solid oral pharmaceutical composition in the form of capsule comprising wet milled diclofenac acid and one or more pharmaceutically acceptable excipients, wherein the diclofenac acid having median particle size of less than 500 nm.

It is to be understood that, the amount of diclofenac acid that can be included in the composition of invention is well known in the art. Preferably, pharmaceutical compositions of invention comprise 35 mg of diclofenac acid or 18 mg of diclofenac acid. The dose of diclofenac acid in the composition of invention can be adjusted according to the requirement.

In another embodiment, a solid oral pharmaceutical composition comprising 18 mg of wet milled diclofenac acid and one or more pharmaceutically acceptable excipients.

In another embodiment, a solid oral pharmaceutical composition comprising 35 mg of wet milled diclofenac acid and one or more pharmaceutically acceptable excipients.

The term “median particle size” means the median particle diameter as determined on an equivalent spherical particle volume basis. Where the term median particle size is used, it indicates that about 50% of all measurable particles measured have a particle size less than the defined median particle size value, and that about 50% of all measurable particles measured have a particle size greater than the defined median particle size value. The median particle size is often written as D₅₀, D(0.50) or D[0.5] or similar. As used herein D₅₀, D(0.50) or D[0.5] or similar shall be taken to mean “median particle size”.

The “median particle size” also refers to the median particle diameter based on mass which means the particle diameter where one half of the mass of particles is contributed by particles with a lesser diameter and one half of the mass of particles is contributed by particles with a greater diameter.

The particle size can be measured using various commonly available methods such as measurement using light (for example light-scattering methods or turbidimetric methods), sedimentation methods (for example pipette analysis using an Andreassen pipette, sedimentation scales, photo-sedimentometers or sedimentation in a centrifugal force), pulse methods (for example Coulter counter), or sorting by means of gravitational or centrifugal force.

In another preferred embodiment, the median particle size is equal to or less than a size selected from the group 1000 nm, 900 nm, 800 nm, 700 nm, 600 nm, 500 nm, 400 nm, 300 nm, 200 nm, 100 nm. In another preferred embodiment, the median particle size is equal to or less than 500 nm.

The term “pharmaceutically acceptable excipient” means any physiologically inert and pharmacologically inactive material known in the art, which is compatible with the physical and chemical characteristics of the active ingredient. Pharmaceutically acceptable excipients include, but are not limited to, polymers, resins, plasticizers, fillers, lubricants, disintegrants, binders, antiadherents, solvents, buffer systems, surfactants, preservatives or pharmaceutical grade dyes or pigments, and viscosity agents.

In another embodiment, one or more pharmaceutically acceptable excipients alone or in any combination are selected from a group of binders, diluents/fillers, lubricants, glidants, disintegrants, sweeteners, flavouring agents, preservatives, buffers, wetting agents, effervescent agents and agents that may form part of a medicament, including a solid dosage form, or other excipients required for other specific drug delivery.

In another embodiment, one excipient can perform others function and the amount of pharmaceutically acceptable excipients employed will depend upon how much active agent is to be used. One excipient can perform multi-functionally.

Binders include, but are not limited to, starches such as potato starch, wheat starch, corn starch; microcrystalline cellulose; celluloses such as hydroxypropyl cellulose, hydroxyethyl cellulose, hydroxypropyl methylcellulose, ethyl cellulose, sodium carboxy methylcellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations thereof and other materials known to one of ordinary skill in the art and mixtures thereof.

Fillers or diluents, which include, but are not limited to sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants may be selected from, but are not limited to, those conventionally known in the art such as magnesium, aluminum or calcium or zinc stearate, stearic acid, polyethylene glycol, glyceryl behenate, mineral oil, sodium stearyl fumarate, stearic acid, hydrogenated vegetable oil and talc.

Glidants include, but are not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

Disintegrants include, but are not limited to, natural Starches, such as Maize Starch, Potato Starch and the like, directly compressible Starches, e.g., Sta-rx® 1500; Modified Starches, e.g., pre-gelatinized starch, Carboxymethyl Starches and Sodium Starch Glycolate, available as Primojel®, Explotab®, Explosol®; and starch derivatives, such as Amylase. Cross-linked polyvinylpyrrolidones, e.g. crospovidones such as Polyplasdone® XL and Kollidon® CL. Alginic acid, Alginates and sodium alginate. Methacrylic acid-divinylbenzene co-polymer (polacrilex resin) and salts thereof, Cross-linked sodium carboxymethylcellulose, available as, e.g., Ac-di-sol®, Primellose®, Pharmacel® XL, Explocel® and Nymcel® ZSX. Additional disintegrants also include microcrystalline cellulose, mannitol, Hydroxypropyl Cellulose, Hydroxypropylmethyl Cellulose, Croscarmellose Sodium, Sodium Starch Glycolate, Polacrillin, Potassium Polyacrylates, such as Carbopol®, Magnesium Aluminium Silicate and Bentonite.

Sweeteners include, but are not limited to, any natural or artificial sweetener including sucrose, xylitol, sodium saccharin, cyclamate, aspartame, and accsulfame K.

Flavouring include, the agents well known to persons skilled in the art and include, but are not limited to Natural extractives (natural flavoring agents) or Artificial flavoring agents which have been approved by the Food and Drug Administration are listed in FDA Regulation 21 CFR 182.20 or 21 CFR 172.515 respectively.

Preservatives include, but are not limited to, methylparaben, ethylpareben, propylparaben, butylparaben, or mixture thereof, benzoic acid, sorbic acid and its salts, other esters of parahydroxybenzoic acid such as butylparaben, alcohols such as ethyl or benzyl alcohol, phenolic chemicals such as phenol, or quarternary compounds such as benzalkonium chloride

Buffers include, but are not limited to, pharmaceutically acceptable salts and acids of acetate, glutamate, citrate, tartrate, benzoate, lactate, histidine or other amino acids, gluconate, phosphate, malate, succinate, formate, propionate, and carbonate.

Wetting agents include, but are not limited to, surfactants, either singly or in admixture. Examples of surfactants include, but are not limited to, poloxamers, heptadecaethylene oxycetanol, lecithins, sorbitol monooleate, polyoxyethylene sorbitol monooleate, polyoxyethylene stearate, polyoxyethylen sorbitan monolaurate, polysorbates for example 20, 40, 60 or 80, sorbitan mono-palmitate, sodium salts of fatty alcohol-sulfates such as sodium lauryl sulfate, sodium dodecylsulfate, sodium salts of sulfosuccinates such as sodium dioctylsulfosuccinate, partially esters of fatty acids with alcohols such as glycerine monostearate, partially esters of fatty acids with sorbitans such as sorbitan monolaurate, partially esters of fatty acids with polyhydroxyethylene sorbitans such as polyethyleneglycol sorbitan monolaurate, -monostearate or -monooleate, ethers of fatty alcohols with polyhydroxyethylene, esters of fatty acids with polyhydroxyethylene, copolymers of ethylenoxide and propylenoxide (Pluronic®) and ethoxylated triglycerides or tyloxapol.

Effervescent agents include, but are not limited to, an effervescent couples such as an organic acid (e.g., citric, tartaric, malic, fumaric, adipic, succinic, and alginic acids and anhydrides and acid salts), or a carbonate (e.g. sodium carbonate, potassium carbonate, magnesium carbonate, sodium glycine carbonate, L-lysine carbonate, and arginine carbonate) or bicarbonate (e.g. sodium bicarbonate or potassium bicarbonate

The pharmaceutical dosage form of the invention can optionally have one or more coatings such as moisture-barrier film coating, sugar coating and other coatings known in the art.

These coating layers comprises one or more excipients selected from the group comprising coating agents, plasticizers, channeling agents, opacifiers, taste-masking agents, fillers, polishing agents, coloring agents, anti-tacking agents and the like.

In another embodiment, a solid oral pharmaceutical composition prepared by wet milling diclofenac acid with povidone alone.

In another embodiment, a solid oral pharmaceutical composition comprising wet milled diclofenac acid and povidone, wherein the diclofenac acid having median particle size of less than 1000 nm.

In another embodiment, a solid oral pharmaceutical composition comprising wet milled diclofenac acid and povidone, wherein the diclofenac acid having median particle size of less than 500 nm.

In another embodiment, a solid oral pharmaceutical composition in the form of Capsules comprising wet milled diclofenac acid and povidone, wherein the diclofenac acid having median particle size of less than 500 nm.

In another embodiment, a solid oral pharmaceutical composition comprising wet milled diclofenac acid and povidone, wherein the diclofenac acid having median particle size of less than 1000 nm, wherein the composition does not contain lactose.

In another embodiment, a solid oral pharmaceutical composition in the form of capsule comprising wet milled diclofenac acid and povidone, wherein the diclofenac acid having median particle size of less than 1000 nm, wherein the composition does not contain lactose.

In another embodiment, a solid oral pharmaceutical composition comprising wet milled diclofenac acid and povidone, wherein the diclofenac acid having median particle size of less than 1000 nm, wherein the composition does not contain sodium lauryl sulphate.

In another embodiment, a solid oral pharmaceutical composition in the form of capsule comprising wet milled diclofenac acid and povidone, wherein the diclofenac acid having median particle size of less than 1000 nm, wherein the composition does not contain sodium lauryl sulphate.

In another embodiment, a solid oral pharmaceutical composition comprising wet milled diclofenac acid and povidone, wherein the diclofenac acid having median particle size of less than 1000 nm, wherein the composition does not contain lactose and sodium lauryl sulphate.

In another embodiment, a solid oral pharmaceutical composition in the form of capsule comprising wet milled diclofenac acid and povidone, wherein the diclofenac acid having median particle size of less than 1000 nm, wherein the composition does not contain lactose and sodium lauryl sulphate.

In another embodiment, a solid oral pharmaceutical composition in the form of capsule comprising 18 mg or 35 mg of wet milled diclofenac acid and povidone, wherein the diclofenac acid having median particle size of less than 1000 nm, wherein the composition does not contain lactose and sodium lauryl sulphate.

In another embodiment, a solid oral pharmaceutical composition comprising wet milled diclofenac acid, povidone and mannitol.

In another embodiment, a solid oral pharmaceutical composition in the form of capsule comprising wet milled diclofenac acid, povidone and mannitol.

In another embodiment, a solid oral pharmaceutical composition comprising wet milled diclofenac acid, povidone and mannitol, wherein the diclofenac acid having median particle size of less than 1000 nm.

In another embodiment, a solid oral pharmaceutical composition in the form of capsule comprising wet milled diclofenac acid, povidone and mannitol, wherein the diclofenac acid having median particle size of less than 1000 nm.

In another embodiment, a solid oral pharmaceutical composition in the form of capsule comprising 18 mg or 35 mg of wet milled diclofenac acid, povidone and mannitol, wherein the diclofenac acid having median particle size of less than 1000 nm.

In another embodiment, a solid oral pharmaceutical composition is in the form of a capsule, granules, a pill, a tablet, a caplet, pellets, a powder or a sachet and more preferably a capsule.

In another embodiment, a solid oral pharmaceutical composition is formed by various processes known in the art but not limited to such as by dry granulation, wet granulation, melt granulation, direct compression, double compression, extrusion spheronization, drug loading, layering and the like. The solvent(s) used in wet granulation include all the solvents well known in the art or the mixtures thereof.

In another embodiment, a process of preparing a solid oral pharmaceutical composition comprising wet milled diclofenac acid and one or more pharmaceutically acceptable excipients comprising:

a) preparing dispersion of diclofenac acid with one or more pharmaceutically acceptable excipients;

b) wet-milling the dispersion of step (a) until median particle size of diclofenac acid reduce to less than 1000 nm;

c) spraying of the dispersion of diclofenac acid of step (b) on one or more pharmaceutically acceptable excipients to form granules;

d) drying the granules of step (c);

e) filling the dried granules of step (d) into capsules.

In another embodiment, a process of preparing a solid oral pharmaceutical composition comprising wet milled diclofenac acid and one or more pharmaceutically acceptable excipients comprising:

a) preparing dispersion of diclofenac acid with povidone;

b) wet-milling the dispersion of step (a) until median particle size of diclofenac acid reduce to less than 1000 nm;

c) spraying of the dispersion of diclofenac acid of step (b) on one or more pharmaceutically acceptable excipients to form granules;

d) drying the granules of step (c);

e) filling the dried granules of step (d) into capsules.

The process of “wet-milling” is carried out by using mills which are known the art like dyno mill. The wet milling involves milling active ingredient (for example diclofenac) with grinding media like zirconium beads in presence of solvents like water, non-aqueous solvents or mixtures thereof. The process of wet milling is carried out until the median particle size of active ingredient (for example diclofenac) is reduced to less than 1000 nm.

It is to be understood for the purpose of invention, the methods which can be employed to determine particle size of wet-milled diclofenac according to the present invention are provided in U.S. Pat. No. 8,679,544.

In another embodiment, a solid oral pharmaceutical composition comprising wet milled diclofenac acid and one or more pharmaceutically acceptable excipients wherein the diclofenac acid having median particle size of less than 1000 nm, such that when the composition tested in-vitro by USP Apparatus I (Basket) method of U.S. Pharmacopoeia at 100 rpm, at 37° C. in 900 ml of 0.05% sodium lauryl sulfate in citric acid solution buffered to pH 5.5 exhibit diclofenac acid release rate of at least 80% by weight within 10 minutes.

In another embodiment, a solid oral pharmaceutical composition comprising wet milled diclofenac acid and one or more pharmaceutically acceptable excipients wherein the diclofenac acid having median particle size of less than 1000 nm, such that when the composition tested in-vitro by USP Apparatus I (Basket) method of U.S. Pharmacopoeia at 100 rpm, at 37° C. in 900 ml of 0.05% sodium lauryl sulfate in citric acid solution buffered to pH 5.5 exhibit diclofenac acid release rate of at least 85% by weight within 20 minutes.

In another embodiment, a solid oral pharmaceutical composition comprising wet milled diclofenac acid and one or more pharmaceutically acceptable excipients wherein the diclofenac acid having median particle size of less than 1000 nm, such that when the composition tested in-vitro by USP Apparatus I (Basket) method of U.S. Pharmacopoeia at 100 rpm, at 37° C. in 900 ml of 0.05% sodium lauryl sulfate in citric acid solution buffered to pH 5.5 exhibit diclofenac acid release rate of at least 85% by weight within 30 minutes.

In another embodiment, a solid oral pharmaceutical composition comprising wet milled diclofenac acid and one or more pharmaceutically acceptable excipients wherein the diclofenac acid having median particle size of less than 1000 nm, such that when the composition tested in-vitro by USP Apparatus I (Basket) method of U.S. Pharmacopoeia at 100 rpm, at 37° C. in 900 ml of 0.05% sodium lauryl sulfate in citric acid solution buffered to pH 5.5 exhibit diclofenac acid release rate of at least 85% by weight within 60 minutes.

In another embodiment, a solid oral pharmaceutical composition comprising wet milled diclofenac acid and one or more pharmaceutically acceptable excipients wherein the diclofenac acid having median particle size of less than 1000 nm, such that when the composition tested in-vitro by USP Apparatus I (Basket) method of U.S. Pharmacopoeia at 100 rpm, at 37° C. in 900 ml of 0.05% sodium lauryl sulfate in citric acid solution buffered to pH 5.5 exhibit diclofenac acid release rate of not more than 87% by weight within 60 minutes.

In another embodiment, a solid oral pharmaceutical composition comprising wet milled diclofenac acid, povidone and mannitol, wherein the diclofenac acid having median particle size of less than 1000 nm, such that when the composition tested in-vitro by USP Apparatus I (Basket) method of U.S. Pharmacopoeia at 100 rpm, at 37° C. in 900 ml of 0.05% sodium lauryl sulfate in citric acid solution buffered to pH 5.5 exhibit diclofenac acid release rate of at least 80% by weight within 10 minutes.

In another embodiment, a solid oral pharmaceutical composition comprising wet milled diclofenac acid and one or more pharmaceutically acceptable excipients wherein the diclofenac acid having median particle size of less than 1000 nm, such that Maximum Plasma Concentration (Cₘₐₓ) and Area Under Curve (AUC) values of the compositions are within the limit of 80% to 125% of Cₘₐₓ and AUC of the marketed composition of diclofenac acid capsule respectively.

In another embodiment, a solid oral pharmaceutical composition comprising wet milled diclofenac acid, povidone and mannitol, wherein the diclofenac acid having median particle size of less than 1000 nm, such that Maximum Plasma Concentration (Cₘₐₓ) and Area Under Curve (AUC) values of the compositions are within the limit of 80% to 125% of Cₘₐₓ and AUC of the marketed composition of diclofenac acid capsule respectively.

In yet another embodiment, the invention comprises a method of treating pain comprising administering the solid oral pharmaceutical composition comprising wet milled diclofenac acid and one or more pharmaceutically acceptable excipients wherein the diclofenac acid having median particle size of less than 1000 nm.

The following examples are illustrative of the present invention, and the examples should not be considered as limiting the scope of this invention in any way, as these examples and other equivalents thereof will become apparent to those versed in the art, in the light of the present disclosure, and the accompanying claims.

### EXAMPLES

| | Quantity |
|---|---|
| Ingredients | (% w/w) |
| Diclofenac Acid | 11.67 |
| Mannitol | 59.85 |
| Microcrystalline Cellulose | 19.68 |
| Croscarmellose Sodium | 4.00 |
| Povidone USP | 4.00 |
| Purified Water | Q.S. |
| Sodium Stearyl Fumarate NF | 0.80 |
| Total Weight | 100.00 |

### Example 1

Procedure:

A. Preparation of Dispersion for Wet milling—
1. Dissolve Povidone in purified water under stiffing.
2. Add Diclofenac acid to step 1 solution under stiffing for sufficient time.

B. Wet Milling of Diclofenac acid Dispersion—
3. Pass the Diclofenac acid dispersion of Step 2 under stiffing through DYNO MILL continuously by using Zirconium oxide beads as grinding media to get milled Diclofenac acid dispersion with diclofenac acid having median particle size of less than 1000 nm
4. Sift the dispersion through sieve before proceeding to drug loading.

C. Drug Loading—
5. Spray the diclofenac acid drug dispersion on mixture of Mannitol, Microcrystalline cellulose, Croscarmellose sodium in fluidized bed processor.
6. Dry the drug loaded granules.
7. Lubricate the drug loaded granules with Sodium stearyl fumarate.

D. Capsule Filling—
8. Fill lubricated blend of step 7 into hard gelatin capsules.

In-Vitro Dissolution Study:

Table 1 given below shows the comparative dissolution profile of Diclofenac acid capsules of Example 1 of the present invention (Test) & ZORVOLEX® 35 mg tablets (Reference) carried out in-vitro by USP Apparatus I (Basket) method of U.S. Pharmacopoeia at 100 rpm, at 37° C. in 900 ml of 0.05% sodium lauryl sulfate in citric acid solution buffered to pH 5.5. The release profile (cumulative % of drug released) is given in Table 1.

TABLE 1

| | Cumulative % Drug Release | |
|---|---|---|
| Time | Example 1 | ZORVOLEX ® 35 mg |
| (Minutes) | (Test) | tablets (Reference) |
| 05 | 48 | 25 |
| 10 | 80 | 56 |
| 20 | 86 | 82 |
| 30 | 86 | 90 |
| 45 | 87 | 91 |
| 60 | 87 | 91 |

1. A solid oral pharmaceutical composition comprising wet milled diclofenac acid and one or more pharmaceutically acceptable excipients wherein the diclofenac acid having median particle size of less than 1000 nm.

2. The solid oral pharmaceutical composition of claim 1, wherein the diclofenac acid has a median particle size of less than 500 nm.

3. The solid oral pharmaceutical composition of claim 1, wherein the composition comprises 35 mg of the diclofenac acid.

4. A solid oral pharmaceutical composition comprising wet milled diclofenac acid having a median particle size of less than 1000 nm, povidone and mannitol.

5. The solid oral pharmaceutical composition of claim 4, wherein the composition does not contain lactose.

6. The solid oral pharmaceutical composition of claim 4, wherein the composition does not contain sodium lauryl sulphate.

7. A process of preparing solid oral pharmaceutical composition comprising wet milled diclofenac acid and one or more pharmaceutically acceptable excipients wherein the diclofenac acid having median particle size of less than 1000 nm comprising
a) preparing dispersion of diclofenac acid with one or more pharmaceutically acceptable excipients;
b) wet-milling the dispersion of step (a) until median particle size of diclofenac acid reduce to less than 1000 nm;
c) spraying of the dispersion of diclofenac acid of step (b) on one or more pharmaceutically acceptable excipients to form granules;
d) drying the granules of step (c);
e) filling the dried granules of step (d) into capsules or compressed into tablets.

8. A solid oral pharmaceutical composition comprising wet milled diclofenac acid and one or more pharmaceutically acceptable excipients wherein the diclofenac acid having median particle size of less than 1000 nm, when tested in-vitro by USP Apparatus I (Basket) method of U.S. Pharmacopoeia at 100 rpm, at 37° C. in 900 ml of 0.05% sodium lauryl sulfate in citric acid solution buffered to pH 5.5 exhibit diclofenac acid release rate of at least 80% by weight within 10 minutes.

9. A solid oral pharmaceutical composition comprising wet milled diclofenac acid and one or more pharmaceutically acceptable excipients wherein the diclofenac acid having median particle size of less than 1000 nm, when tested in-vitro by USP Apparatus I (Basket) method of U.S. Pharmacopoeia at 100 rpm, at 37° C. in 900 ml of 0.05% sodium lauryl sulfate in citric acid solution buffered to pH 5.5 exhibit diclofenac acid release rate of not more than 87% by weight within 60 minutes.

10. The method of treating pain comprising administering the solid oral pharmaceutical composition of claim 1.
